(19) United States
(12) Patent Application Publication
(10) Pub. No.: US 2023/0011681 A1
(43) Pub. Date: Jan. 12, 2023

(54) CANNULA ASSEMBLY INCLUDING A LENS CLEANING ASSEMBLY

(71) Applicant: Covidien LP, Mansfield, MA (US)

(72) Inventor: K V S Manoj Kumar Vadali, Hyderabad (IN)

(21) Appl. No.: 17/368,002

(22) Filed: Jul. 6, 2021

(51) Int. Cl. A61B 1/12; A61B 1/313; A61B 1/00

(52) U.S. Cl. CPC A61B 1/126; A61B 1/3132; A61B 1/00154

(57) ABSTRACT

A cannula assembly includes a housing, an elongate shaft extending from the housing, and a lens cleaning assembly detachably coupled to a distal end portion of the elongate shaft. The elongate shaft defines a lumen therethrough. The lens cleaning assembly includes a base having an annular configuration and defines an aperture in communication with the lumen of the elongate shaft, a cover pivotably coupled to the base, and a spring biasing the cover towards the closed configuration. The cover is transitionable between a closed configuration, in which, the cover closes the aperture of the base, and an open configuration, in which, the cover is spaced apart from the aperture. The cover has a cleaning surface facing the aperture of the base in the closed configuration.

### TECHNICAL FIELD

The disclosure relates to surgical instruments and, more particularly, to a cannula assembly including a lens cleaning assembly configured to remove debris and/or moisture from a lens of an endoscope.

### BACKGROUND

Minimally invasive surgery eliminates the need to cut a large incision in a patient, thereby reducing discomfort, recovery time, and many of the deleterious side effects associated with traditional open surgery. Minimally invasive viewing instruments such as, e.g., laparoscopes and endoscopes, provide viewing of internal tissues and/or organs during the minimally invasive surgery.

Laparoscopic surgery involves the placement of a laparoscope in a small incision in the abdominal wall of a patient, to view the surgical site. Endoscopic surgery involves the placement of an endoscope in a naturally occurring orifice, e.g., mouth, nose, anus, urethra, or vagina, to view the surgical site. Other minimally invasive surgical procedures include video assisted thoracic surgery and cardiovascular surgery conducted through small incisions between the ribs. These procedures also utilize scopes to view the surgical site.

A typical minimally invasive viewing instrument, e.g., a laparoscope or an endoscope, includes a housing, an elongated lens shaft extending from one end of the housing, and a lens that is provided in a distal end of the elongated lens shaft. A camera viewfinder extends from the other end of the housing. A camera is connected to the housing and transmits images sighted through the lens to an external monitor on which the images are displayed. During a surgical procedure, the distal end portion of the elongated lens shaft is extended into the patient, while the proximal end portion of the elongated lens shaft, the housing and the camera viewfinder remain outside the patient. In this manner, the laparoscope/endoscope is positioned and adjusted to view particular anatomical structures in the surgical field on the monitor.

During insertion of an endoscope or a laparoscope into the body and during the surgical procedure, debris, e.g., organic matter and/or moisture, may be deposited on the lens of the scope. The buildup of debris and condensation on the lens impairs visualization of the surgical site, and often necessitates cleaning of the lens.

### SUMMARY

This disclosure describes a cannula assembly that demonstrates a practical approach to meeting the performance requirements and overcoming usability challenges associated with endoscopic or laparoscopic surgery.

In accordance with this disclosure, a cannula assembly includes a housing, an elongate shaft extending from the housing, and a lens cleaning assembly detachably coupled to a distal end portion of the elongate shaft. The elongate shaft defines a lumen therethrough. The lens cleaning assembly includes a base having an annular configuration and defines an aperture in communication with the lumen of the elongate shaft, a cover pivotably coupled to the base, and a spring biasing the cover towards the closed configuration. The cover is transitionable between a closed configuration, in which, the cover closes the aperture of the base, and an open configuration, in which, the cover is spaced apart from the aperture. The cover has a cleaning surface facing the aperture of the base in the closed configuration.

In an aspect, the cover may include a layer of fabric on the cleaning surface.

In another aspect, the layer of fabric may be configured to absorb fluid.

In yet another aspect, the layer of fabric may have a planar surface.

In still yet another aspect, the base may include a lip extending radially inwards from a peripheral portion of the base.

In still yet another aspect, the base and the cover may include a hinge for pivotable transition of the cover between the open and closed configurations.

In an aspect, the spring may be a torsion spring.

In another aspect, the base may include a second surface opposite of the cleaning surface. The second surface may have an atraumatic configuration.

In yet another aspect, the base of the lens cleaning assembly may be formed of a flexible material.

In still yet another aspect, the cover in the closed configuration may define an acute angle with respect to a longitudinal axis defined by the elongate shaft.

In accordance with this disclosure, a surgical kit includes an endoscope including an elongate member including a lens at a distal end of the elongate member and a cannula assembly. The cannula assembly includes a housing, an elongate shaft extending from the housing, and a lens cleaning assembly supported on a distal end portion of the elongate shaft. The elongate shaft defines a lumen to receive the endoscope. The lens cleaning assembly includes a base defining an aperture configured to receive the endoscope therethrough, a cover coupled to the base and transitionable between a closed configuration, in which, the cover closes the aperture of the base, and an open configuration, in which, the cover is spaced apart from the aperture, and a spring biasing the cover towards the closed configuration. The cover includes a cleaning surface configured to engage the lens of the endoscope to remove debris from a surface of the lens.

In an aspect, the cleaning surface may include a layer of fabric.

In another aspect, the layer of fabric may be configured to absorb fluid.

In yet another aspect, the distal end of the endoscope and the cleaning surface of the lens cleaning assembly may be tapered.

In still yet another aspect, the cleaning surface of the lens cleaning assembly may be configured for a planar contact when the lens of the endoscope is advanced through the lumen of the cannula assembly.

In still yet another aspect, the cover of the lens cleaning assembly may include a distal portion having an atraumatic configuration.

In still yet another aspect, the cover and the base may include a hinge.

In still yet another aspect, the lens cleaning assembly may be detachably mounted on the distal end portion of the elongate shaft.

In an aspect, the spring may be a torsion spring.

### BRIEF DESCRIPTION OF DRAWINGS

The above and other aspects and features of this disclosure will become more apparent in view of the following detailed description when taken in conjunction with the accompanying drawings wherein like reference numerals identify similar or identical elements.

FIG. 1 is a perspective view of a cannula assembly in accordance with the disclosure, illustrating use with an endoscope;

FIG. 2 is an enlarged view of the indicated area of detail of FIG. 1;

FIG. 3 is an exploded perspective view of a lens cleaning assembly of FIG. 1 with parts separated;

FIG. 4 is a side view of the cannula assembly and the endoscope of FIG. 1, illustrating use in a body cavity;

FIGS. 5 and 6 are partial side views of a distal end portion of the cannula assembly of FIG. 1, illustrating the lens cleaning assembly in a closed configuration based on a relative position of the endoscope;

FIG. 7 is a partial perspective view of the cannula assembly of FIG. 2, illustrating the lens cleaning assembly transitioning to an open configuration; and

FIGS. 8 and 9 are partial side views of a distal end portion of the cannula assembly of FIG. 1, illustrating transitioning of the lens cleaning assembly to the open configuration.

### DETAILED DESCRIPTION

The cannula assembly disclosed herein is described in detail with reference to the drawings, in which like reference numerals designate identical or corresponding elements in each of the several views.

As used herein, the term “distal” refers to the portion that is being described which is farther from a user, while the term “proximal” refers to the portion that is being described which is closer to a user. In addition, the terms parallel and perpendicular are understood to include relative configurations that are substantially parallel and substantially perpendicular up to about + or −10 degrees from true parallel and true perpendicular. Further, to the extent consistent, any or all of the aspects detailed herein may be used in conjunction with any or all of the other aspects detailed herein.

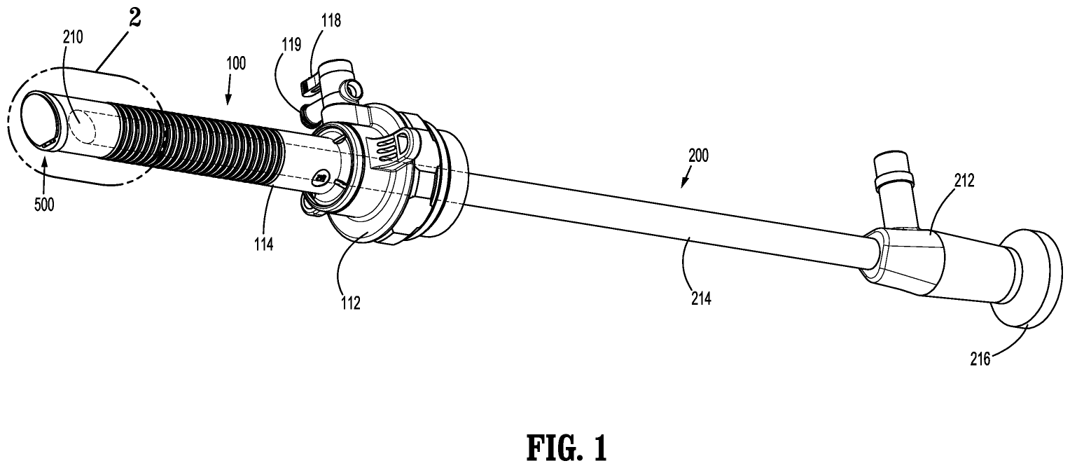

With reference to FIG. 1, an exemplary cannula assembly in accordance with the disclosure is shown generally as a cannula assembly 100. The cannula assembly 100 is configured to permit access to, e.g., an insufflated abdominal cavity, during a minimally invasive surgical procedure to permit the introduction of a surgical object for performing various surgical tasks on internal organs within the cavity. The surgical object may be an endoscope 200 or a surgical instrument such as laparoscopic or endoscopic clip appliers, obturators, graspers, dissectors, retractors, staplers, laser probes, photographic devices, tubes, electro-surgical devices and the like. In particular, the cannula assembly 100 includes a lens cleaning assembly 500 configured to remove debris and/or moisture from a lens 210 of the endoscope 200, as will be described below.

The endoscope 200 includes a housing 212 and an elongated tubular shaft 214 extending distally from the housing 212 and terminating in the lens 210. The elongate tubular shaft 214 may be rigid, semi-rigid, or flexible. The housing 212 includes a viewfinder 216 adapted to sight images of a surgical field in the patient, e.g., an abdominal cavity, thoracic cavity, etc., as the position of the endoscope 200 is adjusted to view a particular anatomical structure in the surgical field. A camera (not shown) is adapted to receive images of the surgical field sighted through the lens 210 and transmit the images to, e.g., an external monitor, on which the images of the surgical field are displayed. That is, a visual display device converts the optical signal into a video signal to produce a video image on the monitor (or for storage on select media). Accordingly, the monitor enables a clinician to view the anatomical structure in the surgical field inside the patient as the surgical procedure is carried out using minimally invasive or endoscopic surgical instruments. Throughout the surgical procedure, condensation, smoke particles, and biological tissue or matter tend to contact and build up on the lens 210 of the endoscope 200. This tends to obscure the images of the surgical field as they are displayed on the monitor. To this end, the cannula assembly 100 includes the lens cleaning assembly 500 that enables cleaning of the lens 210 during the surgical procedure to maintain a clear image without having to remove the endoscope 200 from the patient's body, which, in turn, reduces the duration of surgery.

The cannula assembly 100 generally includes a cannula housing 112 and an elongate shaft 114 extending from the cannula housing 112. The elongate shaft 114 may be rigid, semi-rigid, or flexible. The cannula housing 112 is dimensioned for engagement by the clinician and may include one or more internal seals adapted to establish a seal about a surgical object introduced therethrough. The cannula housing 112 also may include an insufflation valve 118 with a fluid connector 119 (e.g., a luer connector) for connecting to a source of insufflation fluids (not shown) for delivery within a surgical site, e.g., the abdominal cavity. A longitudinal lumen defined by the elongate shaft 114 is also in fluid communication with the insufflation connector 118 to convey insufflation fluids into the abdominal cavity to establish and/or maintain the pneumoperitoneum.

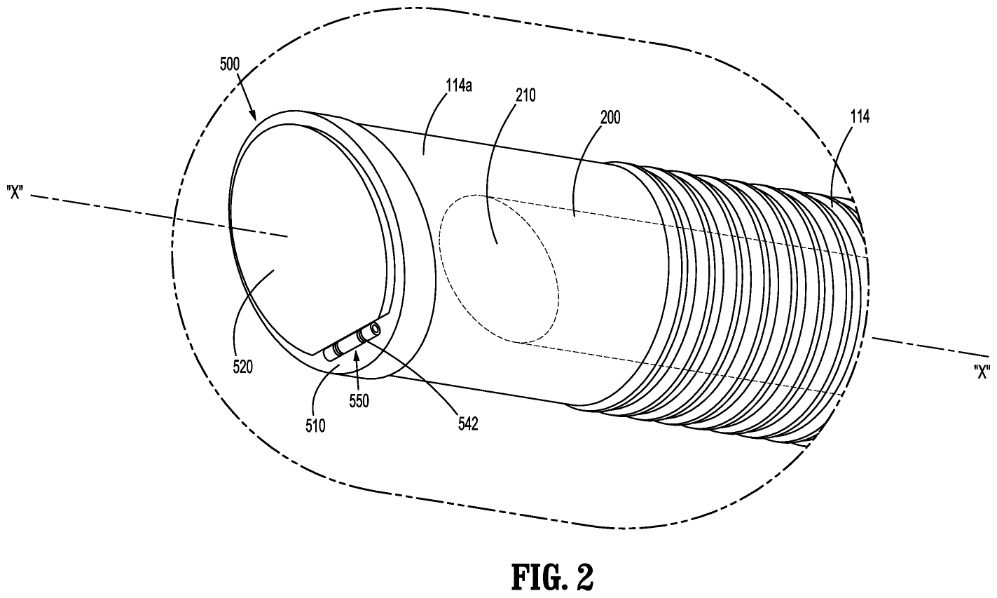

FIG. 2 illustrates a distal end portion 114a of the elongate shaft 114 of the cannula assembly 100 (FIG. 1). The distal end portion 114a includes a lens cleaning assembly 500 detachably coupled thereto. The lens cleaning assembly 500 includes a base 510, a cover 520 pivotably coupled to the base 510 by a hinge 550, and springs 542 to bias the cover 520 towards a closed position. The distal end portion 114a of the elongate shaft 114 of the cannula assembly 100 may be tapered to facilitate insertion into an anatomical structure of a patient. To this end, the base 510 of the lens cleaning assembly 500 may have the same contour or tapered profile as the distal end portion 114a of the elongate shaft 114. The base 510 defines an acute angle with respect to a longitudinal axis “X-X” defined by the elongate shaft 114. In addition, the base 510 may be formed of a flexible or resilient material such as, a polymer or gel, to be detachably coupled to the distal end portion 114a of the elongate shaft 114. However, it is contemplated that the base 510 may be coupled to the distal end portion 114a through, e.g., snap fit or bayonet coupling.

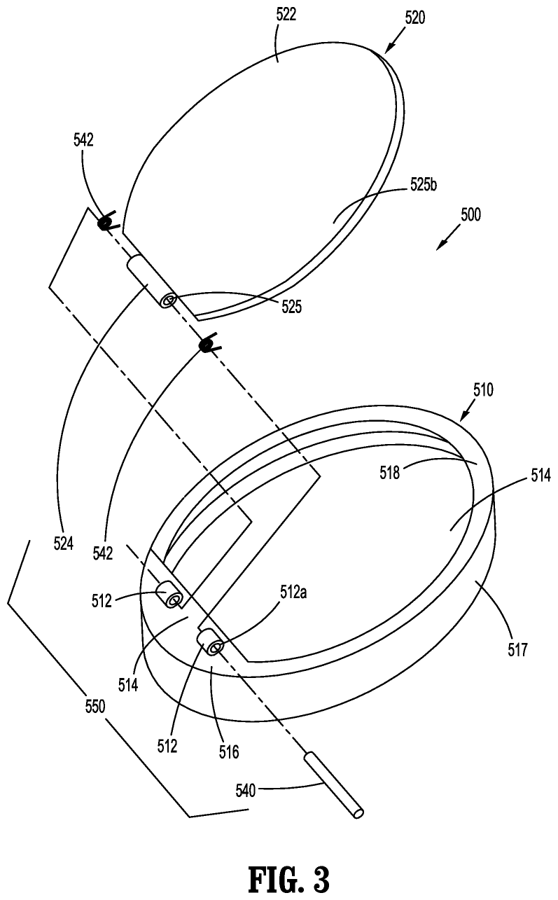

FIG. 3 illustrates the base 510 having an annular configuration defining an aperture 514 therethrough. The base 510 includes a lip portion 518 that extends radially inwards from a peripheral portion 517 of the base 510. The lip portion 518 serves as a stopper when the lens cleaning assembly 500 is coupled to the elongate shaft 114 of the cannula 100, as well as to inhibit the cover 520 from extending into the aperture 514 In addition, the base 510 further includes a distal surface 516 including a pair of sleeves 512 forming a portion of the hinge assembly 550, as will be described below.

The hinge assembly 550 pivotably couples the cover 520 to the base 510. In particular, the pair of sleeves 512 of the base 510 defines a gap 514 and bores 512a dimensioned to receive the pivot pin 540. The cover 520 has a covering portion 522 configured to close the aperture 514 of the base 510. The covering portion 522 has a sleeve 524 that is part of the hinge assembly 550. The sleeve 524 is dimensioned to be received within the gap 514 defined by the pair of sleeves 512 of the base 510. The sleeve 524 defines a bore 525 in alignment with the bores 512a of the pair of sleeves 512 to receive the pivot pin 540 such that the cover 520 is pivotable relative to the base 510. In addition, the lens cleaning assembly 500 may further include springs 542 (e.g., torsion springs) to bias the cover 520 towards a closed configuration, in which, the cover 520 engages the lip portion 518 of the base 510 and closes the aperture 514 of the base 510.

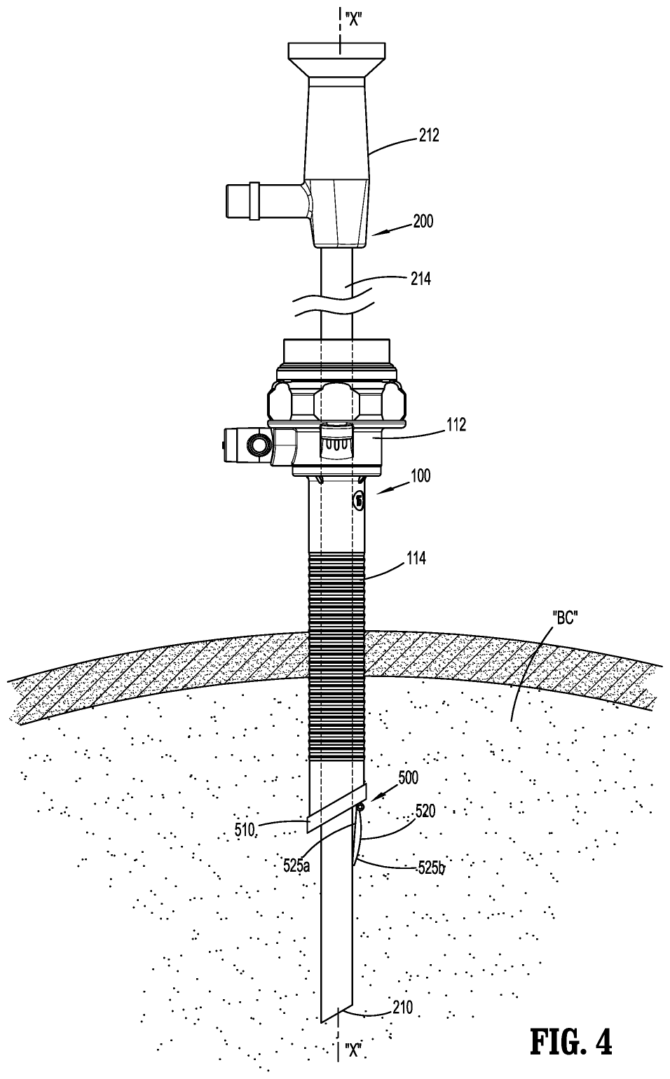

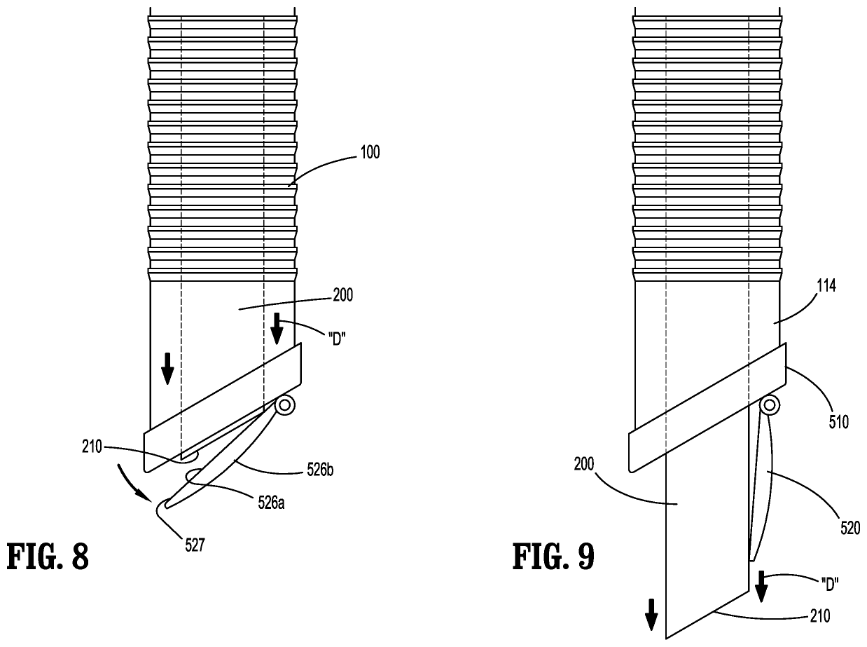

The cover 520 further includes first and second portions 525a, 525b (FIG. 4). In particular, the first portion 525a includes a cleaning surface 526a (FIG. 8) that includes, e.g., a layer of fabric 527 (FIG. 8), to wipe the lens 210 of the endoscope 200, as will be described below. In an aspect, the cleaning surface 526a may define a plane for planar contact with the lens 210 of the endoscope 200. The second portion 525b (FIG. 8) may include an atraumatic surface having, e.g., an arcuate profile, to inhibit trauma to tissue during insertion or manipulation of the cannula assembly 100 in a body cavity “BC” (FIG. 4) of a patient.

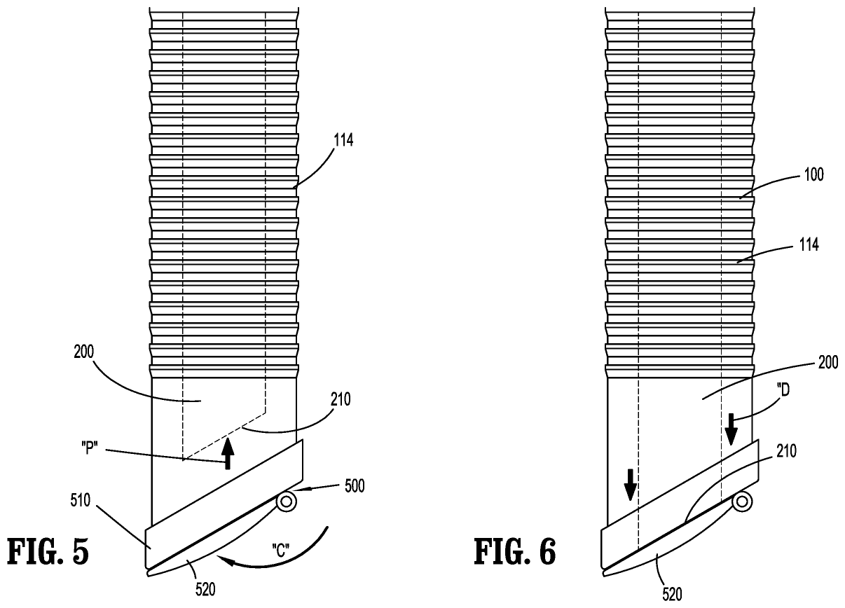

FIG. 5 illustrates the cover 520 of the lens cleaning assembly 500 biased towards a closed configuration (in the direction of an arrow “C”) such that the cover 520 closes the aperture 514 (FIG. 3) of the base 510 of the lens cleaning assembly 500. However, as the endoscope 200 is advanced (in the direction of arrows “D”), (FIG. 6) the lens 210 of the endoscope 200 engages the cleaning surface 526a (FIG. 8) of the cover 520 and the cover 520 transitions towards the open configuration (in the direction of an arrow “O”). The lens 210 is wiped against the cleaning surface 526a of the cover 520 as the lens 210 extends through the aperture 514 of the base 510. In this manner, the debris and/or fluid on the lens 210 of the endoscope 200 may be safely removed therefrom. As described hereinabove, the cleaning surface 526a may include a layer of fabric 527 (FIG. 8). In an aspect, the layer of fabric 527 may be configured to absorb fluid or moisture on the lens 210. In another aspect, the layer of fabric 527 may further include bristles to facilitate removal of debris from the lens 210. A distal end of the endoscope 200 is tapered. The lens cleaning assembly 500 may be tapered to achieve a maximum contact between the cleaning surface 526a of the cover 520 and a surface of the lens 210. For example, the lens cleaning assembly 500 may be tapered to provide a planar contact between the cleaning surface 526a of the cover 520 and the surface of the lens 210 while the lens 210 is being advanced.

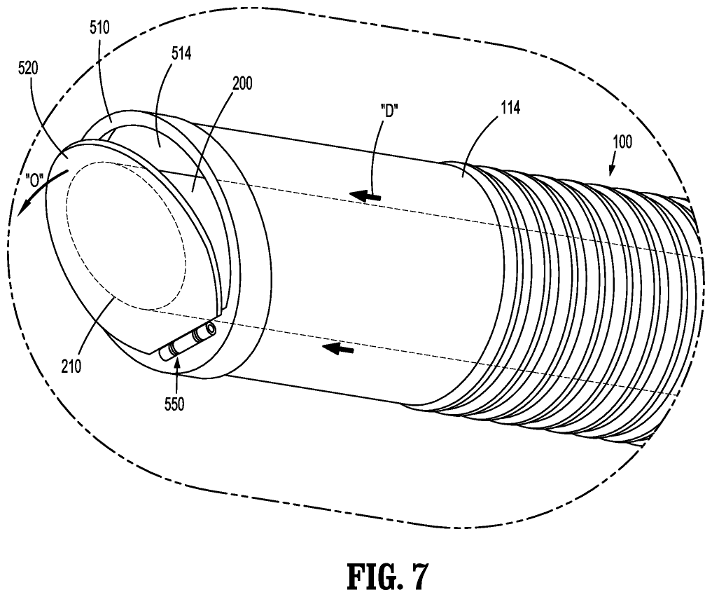

In use, the cannula assembly 100 is positioned through tissue in a desired orientation. Thereafter, the endoscope 200 may be inserted through the cannula assembly 100 such that the endoscope 200 is directed to the surgical site, as shown in FIG. 4. When the lens 210 of the endoscope 200 is obscured by the fluids or debris, the clinician may retract the lens 210 in the direction of an arrow “P” (FIG. 5) such that the lens 210 is disposed within the elongate shaft 114 of the cannula assembly 100 and the cover 520 transitions to the closed configuration under the influence of the springs 542. At this time, as shown in FIGS. 6-8, the endoscope 200 may be advanced in the direction of the arrows “D”, such that the lens 210 is wiped against the cleaning surface 526a (FIG. 8) of the cover 520. In this manner, the clinician may improve visibility of the lens 210 without extracting the endoscope 200 from the surgical site, which, in turn, may save operating time and eliminate additional trauma to the patient. Thereafter, as shown in FIG. 9, the clinician may resume the surgical procedure.

It is also envisioned that the lens cleaning assembly 500 may be adapted for use with a robotic surgical system. It is further contemplated that the elongate shaft 114 of the cannula assembly 100 may further include a fluid port positioned adjacent the cannula housing 112. It is further contemplated that the cannula assembly 100 may further include a fluid port that is adapted to be coupled to a source of inflation fluids to inflate an expandable balloon on the elongate shaft 114 to seal the expandable balloon against tissue to improve securement of the cannula assembly 100 to tissue and to retain insufflation fluid within the abdominal cavity. The expandable balloon expands radially outwardly upon introduction of inflation fluids through the fluid port.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A cannula assembly comprising:
a housing;
an elongate shaft extending from the housing, the elongate shaft defining a lumen therethrough; and
a lens cleaning assembly detachably coupled to a distal end portion of the elongate shaft, the lens cleaning assembly including:
a base having an annular configuration and defining an aperture in communication with the lumen of the elongate shaft;
a cover pivotably coupled to the base, the cover transitionable between a closed configuration, in which, the cover closes the aperture of the base, and an open configuration, in which, the cover is spaced apart from the aperture, the cover having a cleaning surface facing the aperture of the base in the closed configuration; and
a spring biasing the cover towards the closed configuration.

2. The cannula assembly according to claim 1, wherein the cover includes a layer of fabric on the cleaning surface.

3. The cannula assembly according to claim 2, wherein the layer of fabric is configured to absorb fluid.

4. The cannula assembly according to claim 2, wherein the layer of fabric has a planar surface.

5. The cannula assembly according to claim 1, wherein the base includes a lip extending radially inwards from a peripheral portion of the base.

6. The cannula assembly according to claim 1, wherein the base and the cover include a hinge for pivotable transition of the cover between the open and closed configurations.

7. The cannula assembly according to claim 1, wherein the spring is a torsion spring.

8. The cannula assembly according to claim 1, wherein the base includes a second surface opposite of the cleaning surface, the second surface having an atraumatic configuration.

9. The cannula assembly according to claim 1, wherein the base of the lens cleaning assembly is formed of a flexible material.

10. The cannula assembly according to claim 1, wherein the closed configuration of the cover defines an acute angle with respect to a longitudinal axis defined by the elongate shaft.

11. A surgical kit comprising:
an endoscope including an elongate member including a lens at a distal end of the elongate member; and
a cannula assembly including
a housing;
an elongate shaft extending from the housing, the elongate shaft defining a lumen to receive the endoscope; and
a lens cleaning assembly supported on a distal end portion of the elongate shaft, the lens cleaning assembly including:
a base defining an aperture configured to receive the endoscope therethrough;
a cover coupled to the base and transitionable between a closed configuration, in which, the cover closes the aperture of the base, and an open configuration, in which, the cover is spaced apart from the aperture, the cover including a cleaning surface configured to engage the lens of the endoscope to remove debris from a surface of the lens; and
a spring biasing the cover towards the closed configuration.

12. The surgical kit according to claim 11, wherein the cleaning surface includes a layer of fabric.

13. The surgical kit according to claim 12, wherein the layer of fabric is configured to absorb fluid.

14. The surgical kit according to claim 11, wherein the distal end of the endoscope and the cleaning surface of the lens cleaning assembly are tapered.

15. The surgical kit according to claim 11, wherein the cleaning surface of the lens cleaning assembly is configured for a planar contact when the lens of the endoscope is advanced through the lumen of the cannula assembly.

16. The surgical kit according to claim 11, wherein the cover of the lens cleaning assembly includes a distal portion having an atraumatic configuration.

17. The surgical kit according to claim 11, wherein the cover and the base include a hinge.

18. The surgical kit according to claim 11, wherein the lens cleaning assembly is detachably mounted on the distal end portion of the elongate shaft.

19. The surgical kit according to claim 11, wherein the spring is a torsion spring.
